(19) United States
(12) Patent Application Publication
(10) Pub. No.: US 2010/0121155 A1
(43) Pub. Date: May 13, 2010

(54) Minimally Invasive Tissue Modification Systems With Integrated Visualization

(76) Inventors: Xiaolong OuYang, Palo Alto, CA (US); James S. Cybulski, Menlo Park, CA (US); Fred R. Seddiqui, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/269,775

(22) Filed: Nov. 12, 2008

(51) Int. Cl. A61B 17/295; A61B 1/06

(52) U.S. Cl. 600/249; 606/83

(57) ABSTRACT

Aspects of the invention include minimally invasive tissue modification systems. Embodiments of the systems include a minimally invasive access device having a proximal end, a distal end and an internal passageway. Also part of the system is an elongated tissue modification device having a proximal end and a distal end. The tissue modification device is dimensioned to be slidably moved through the internal passageway of the access device. The tissue modification device includes a tissue modifier. Positioned among the distal ends of the devices are a visualization element and an illumination element. Also provided are methods of using the systems in tissue modification applications, as well as kits for practicing the methods of the invention.

### INTRODUCTION

Many pathological conditions in the human body may be caused by enlargement, movement, displacement and/or a variety of other changes of bodily tissue, causing the tissue to press against (or “impinge on”) one or more otherwise normal tissues or organs. For example, a cancerous tumor may press against an adjacent organ and adversely affect the functioning and/or the health of that organ. In other cases, bony growths (or “bone spurs”), arthritic changes in bone and/or soft tissue, redundant soft tissue, or other hypertrophic bone or soft tissue conditions may impinge on nearby nerve and/or vascular tissues and compromise functioning of one or more nerves, reduce blood flow through a blood vessel, or both. Other examples of tissues which may grow or move to press against adjacent tissues include ligaments, tendons, cysts, cartilage, scar tissue, blood vessels, adipose tissue, tumor, hematoma, and inflammatory tissue.

The intervertebral disc 10 is composed of a thick outer ring of cartilage (annulus) 12 and an inner gel-like substance (nucleus pulposus) 14. A three-dimensional view of an intervertebral disc 10 is provided in FIG. 1. The annulus 12 contains collagen fibers that form concentric lamellae 16 that surround the nucleus 14 and insert into the endplates of the adjacent vertebral bodies. The nucleus pulposus comprises proteoglycans entrapped by a network of collagen and elastin fibers which has the capacity to bind water. When healthy, the intervertebral disc keeps the spine flexible and serves as a shock absorber by allowing the body to accept and dissipate loads across multiple levels in the spine.

With respect to the spine and intervertebral discs, a variety of medical conditions can occur in which it is desirable to ultimately surgically remove at least some of if not all of an intervertebral disc. As such, a variety of different conditions exist where partial or total disc removal is desirable.

One such condition is disc herniation. Over time, the nucleus pulposus becomes less fluid and more viscous as a result of age, normal wear and tear, and damage caused from an injury. The proteoglycan and water from within the nucleus decreases which in turn results in the nucleus drying out and becoming smaller and compressed. Additionally, the annulus tends to thicken, desiccate, and become more rigid, lessening its ability to elastically deform under load and making it susceptible to disc fissures.

A fissure occurs when the fibrous components of the annulus become separated in particular areas, creating a tear within the annulus. The most common type of fissure is a radial fissure in which the tear is perpendicular to the direction of the fibers. A fissure associated with disc herniation generally falls into three types of categories: 1) contained disc herniation (also known as contained disc protrusion); 2) extruded disc herniation; and 3) sequestered disc herniation (also known as a free fragment.) In a contained herniation, a portion of the disc protrudes or bulges from a normal boundary of the disc but does not breach the outer annulus fibrosis. In an extruded herniation, the annulus is disrupted and a segment of the nucleus protrudes/extrudes from the disc. However, in this condition, the nucleus within the disc remains contiguous with the extruded fragment. With a sequestered disc herniation, a nucleus fragment separates from the nucleus and disc.

As the posterior and posterolateral portions of the annulus are most susceptible to herniation, in many instances, the nucleus pulposus progresses into the fissure from the nucleus in a posteriorly or posterolateral direction. Additionally, biochemicals contained within the nucleus pulposus may escape through the annulus causing inflammation and irritating adjacent nerves. Symptoms of a herniated disc generally include sharp back or neck pain which radiates into the extremities, numbness, muscle weakness, and in late stages, paralysis, muscle atrophy and bladder and bowel incontinence.

Conservative therapy is the first line of treating a herniated disc which includes bed rest, medications to reduce inflammation and pain, physical therapy, patient education on proper body mechanics and weight control.

If conservative therapy offers no improvement then surgery is recommended. Open discectomy is the most common surgical treatment for ruptured or herniated discs. The procedure involves an incision in the skin over the spine to remove the herniated disc material so it no longer presses on the nerves and spinal cord. Before the disc material is removed, some of the bone from the affected vertebra may be removed using a laminotomy or laminectomy to allow the surgeon to better see the area. As an alternative to open surgery, minimally invasive techniques have been rapidly replacing open surgery in treating herniated discs. Minimally invasive surgery utilizes small skin incisions, thereby minimizing the damaging effects of large muscle retraction and offering rapid recovery, less post-operative pain and small incisional scars.

### SUMMARY

Aspects of the invention include minimally invasive tissue modification systems. Embodiments of the systems include a minimally invasive access device having a proximal end, a distal end and an internal passageway. The distal end of the access device includes an illumination element. Also part of the system is an elongated tissue modification device having a proximal end and a distal end. The tissue modification device is dimensioned to be slidably moved through the internal passageway of the access device. The tissue modification device includes a tissue modifier and a visualization element integrated at the distal end. Also provided are methods of using the systems in tissue modification applications, as well as kits for practicing the methods of the invention.

### BRIEF DESCRIPTION OF THE FIGURES

FIG. 1 provides a three-dimensional view of an intervertebral disc according to one embodiment of the invention.

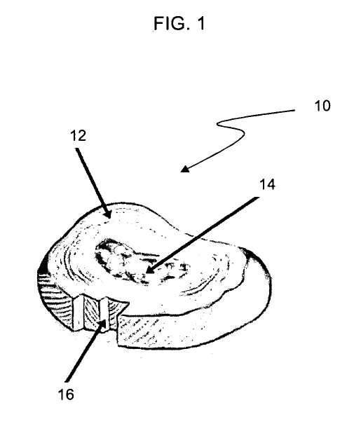

FIG. 2 provides a view of a rongeur modification device according to one embodiment a system of the invention.

FIG. 3 provides views of an access device of a system of the invention configured to be employed with the rongeur modification device according to FIG. 2.

FIG. 4 provides views of an access device of a system of the invention in which the access device is made up of a translucent material and includes a reflective outer coating.

### DETAILED DESCRIPTION

Aspects of the invention include minimally invasive tissue modification systems. Embodiments of the systems include a minimally invasive access device having a proximal end, a distal end and an internal passageway. The distal end of the access device includes an illumination element. Also part of the system is an elongated tissue modification device having a proximal end and a distal end. The tissue modification device is dimensioned to be slidably moved through the internal passageway of the access device. The tissue modification device includes a tissue modifier and a visualization element integrated at the distal end. Also provided are methods of using the systems in tissue modification applications, as well as kits for practicing the methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, embodiments of the minimally invasive tissue modification systems and components thereof are reviewed first in greater detail, followed by a review of embodiments of methods of using the devices.

### Minimally Invasive Tissue Modification Systems

As summarized above, aspects of the invention include minimally invasive tissue modification systems. The systems of the invention are minimally invasive, such that they may be introduced to an internal target site of a patient, e.g., a spinal location that is near or inside of an intervertebral disc, through a minimal incision, e.g., an incision that is less than the size of an incision employed for an access device having a outer diameter of 20 mm or larger, e.g., less than 75% the size of such an incision, such as less than 50% of the size of such an incision, or smaller.

Tissue modification systems of the invention include both an access device and an elongated tissue modification device. The access device is a device having a proximal end and a distal end and an internal passageway extending from the proximal end to the distal end. Similarly, the elongated tissue modification device has a proximal end and a distal end and is dimensioned to be slidably moved through the internal passageway of the access device.

Aspects of the invention include a visualization element and an illumination element that are positioned among the distal ends of the access device and the elongated member. The phrase “among the distal ends of the access device and elongated member” means that between the two distal ends, there is positioned at least one visualization element and at least one illumination element. By “located among the distal ends” is meant that the item of interest (e.g., the visualization element, the illumination element) is present at the distal end of the elongate member and/or access device, or near the distal end of the elongate member and/or access device, e.g., within 10 mm or closer to the distal end, such as within 5 mm or closer to the distal end and including within 3 mm or closer to the distal end.

In certain embodiments, the visualization element and illumination are positioned at the distal end of the same member of the system, e.g., at the distal end of the elongated member or at the distal end of the access device. In yet other embodiments, the visualization and illumination elements are present on different components of the device, e.g., where the visualization element is on the elongated member and the illumination element is on the access device, or vice versa. For ease of description, the systems of the invention will now be further described in terms of embodiments where the visualization element is present on the elongated structure and the illumination element is present on the access device.

### Access Devices

Access devices of the invention are elongated elements having an internal passageway that are configured to provide access to a user e.g., a health care professional, such as a surgeon, from an extra-corporeal location to an internal target tissue site, e.g., a location near or in the spine or component thereof, e.g., near or in an intervertebral disc, inside of the disc, etc., through a minimally invasive incision. Access devices of the invention may be cannulas, components of retractor tube systems, etc. As the access devices are elongate, they have a length that is 1.5 times or longer than their width, such as 2 times or longer than their width, including 5 or even 10 times or longer than their width, e.g., 20 times longer than its width, 30 times longer than its width, or longer.

Where the access devices are configured to provide access through a minimally invasive incision, the longest cross-sectional outer dimension of the access devices (for example, the outer diameter of a tube shaped access device, including wall thickness of the access device, which may be a port or cannula in some instances) ranges in certain instances from 5 mm to 50 mm, such as 10 to 20 mm. With respect to the internal passageway, this passageway is dimensioned to provide passage of the tools, e.g., imaging devices, tissue modifiers, etc., from an extra-corporeal site to the internal target tissue location. In certain embodiments, the longest cross-sectional dimension of the internal passageway, e.g., the inner diameter of a tubular shaped access device, ranges in length from 5 to 30 mm, such as 5 to 25 mm, including 5 to 20 mm, e.g., 7 to 18 mm. Where desired, the access devices are sufficiently rigid to maintain mechanical separation of tissue, e.g., muscle, and may be fabricated from any convenient material. Materials of interest from which the access devices may be fabricated include, but are not limited to: metals, such as stainless steel and other medical grade metallic materials, plastics, and the like.

Aspects of the access devices of the invention include the presence of one or more illumination elements that are positioned at the distal end of the access device. By “positioned at the distal end” is meant that the illumination element(s) is present at the distal end of the access device, or near the distal end of the access device, e.g., within 10 mm or closer to the distal end, such as within 5 mm or closer to the distal end and including within 3 mm or closer to the distal end of the access device. A variety of different types of lights sources may be employed as illumination elements, so long as their dimensions are such that they can be positioned at the distal end of the access device. The light sources may be light emitting diodes configured to emit light of the desired wavelength range, or optical conveyance elements, e.g., optical fibers, configured to convey light of the desired wavelength range from a location other than the distal end of the access device, e.g., a location at the proximal end of the access device, to the distal end of the access device. Where desired, the light sources may include a diffusion element to provide for uniform illumination of the target tissue site. Any convenient diffusion element may be employed, including but not limited to a translucent cover or layer (fabricated from any convenient translucent material) through which light from the light source passes and is thus diffused. In certain instances, two or more distinct types of light sources may be present at the distal end, e.g., both LED and fiber optic light sources. The light sources may be integrated with the access device, e.g., may be configured relative to the access device such that the light source is a component of the access device, and cannot be removed from the remainder of the access device without significantly compromising the structure of the access device. As such, the integrated illumination element of these embodiments is not readily removable from the remainder of the access device, such that the illumination element and remainder of the access device form an inter-related whole. The light sources may include a conductive element, e.g., wire, optical fiber, etc., which runs the length of the access device to provide for control of the light source from a location outside the body, e.g., an extracorporeal control device. In certain instances, the access device is fabricated from a translucent material which conducts light from a source apart from the distal end, e.g., from the proximal end, to the distal end. Where desired, a reflective coating may be provided on the outside of the translucent access device to internally reflect light provided from a remote source, e.g., such as an LED at the proximal end, to the distal end of the device. Any convenient reflective coating material may be employed. In those embodiments of the invention where the system includes two or more illumination elements, the illumination elements may emit light of the same wavelength or they may be spectrally distinct light sources, where by “spectrally distinct” is meant that the light sources emit light at wavelengths that do not substantially overlap, such as white light and near-infra-red light, such as the spectrally distinct light sources described in copending U.S. application Ser. No. ______ titled “Minimally Invasive Imaging Device” filed on even date herewith (Attorney docket no. AXIS-003); the disclosure of which is herein incorporated by reference.

### Tissue Modification Devices

Tissue modification devices of the invention are elongate members having a proximal and distal end, where the elongate members are dimensioned to be slidably moved through the internal passageway of the access device. As this component of the systems is elongate, it has a length that is 1.5 times or longer than its width, such as 2 times or longer than its width, including 5 or even 10 times or longer than its width, e.g., 20 times longer than its width, 30 times longer than its width, or longer. When designed for use in IVD procedures, the elongate member is dimensioned to access an intervertebral disc. By “dimensioned to access an intervertebral disc” is meant that at least the distal end of the device has a longest cross-sectional dimension that is 10 mm or less, such as 8 mm or less and including 7 mm or less, where in certain embodiments the longest cross-sectional dimension has a length ranging from 5 to 10 mm, such as 6 to 9 mm, and including 6 to 8 mm. The elongate member may be solid or include one or more lumens, such that it may be viewed as a catheter. The term “catheter” is employed in its conventional sense to refer to a hollow, flexible or semi-rigid tube configured to be inserted into a body. Catheters of the invention may include a single lumen, or two or more lumens, e.g., three or more lumens, etc, as desired. Depending on the particular embodiment, the elongate members may be flexible or rigid, and may be fabricated from any convenient material.

Where desired, the devices may include a handle or analogous control structure connected to the proximal end of the elongated member and a working element connected to the distal end of the elongated member. The handle, which may include gripping portions or other convenient structures, is operably connected to the tissue modifier at the distal end of the device so that manipulations performed on the handle, for example manually by a surgeon or by a computer controlled actuator, are translated to the tissue modifier to cause the tissue modifier to move in a manner that provides for desired mechanical tissue modification.

The tissue modifier at the distal end may vary considerably. Examples of tissue modifiers that may be present at the distal end include, but are not limited to: mechanical tissue modifiers, such as rongeur forceps, a curette, a scalpel, one or more cutting blades, a scissors, a forceps, a probe, a rasp, a file, an abrasive element, one or more small planes, a rotary powered mechanical shaver, a reciprocating powered mechanical shaver, a powered mechanical burr, etc.; coagulators, electrosurgical electrodes, active agent delivery devices, e.g., needles, etc.

Integrated at the distal end of the tissue modification device, e.g., near to or part of the tissue modification element, is a visualization element. Of interest as visualization elements are imaging sensors. Imaging sensors of interest are miniature in size so as to be integrated with the tissue modification device at the distal end. Miniature imaging sensors of interest are those that, when integrated at the distal end of an elongated structure along with an illumination source, e.g., such as an LED as reviewed below, can be positioned on a probe having a longest cross section dimension of 6 mm or less, such as 5 mm or less, including 4 mm or less, and even 3 mm or less. In certain embodiments, the miniature imaging sensors have a longest cross-section dimension (such as a diagonal dimension) of 5 mm or less, such 3 mm or less, where in certain instances the sensors may have a longest cross-sectional dimension ranging from 2 to 3 mm. In certain embodiments, the miniature imaging sensors have a cross-sectional area that is sufficiently small for its intended use and yet retain a sufficiently high matrix resolution. Certain imaging sensors of the invention have a cross-sectional area (i.e. an x-y dimension, also known as packaged chip size) that is 2 mm×2 mm or less, such as 1.8 mm×1.8 mm or less, and yet have a matrix resolution of 400×400 or greater, such as 640×480 or greater. Imaging sensors of interest are those that include a photosensitive component, e.g., array of photosensitive elements, coupled to an integrated circuit, where the integrated circuit is configured to obtain and integrate the signals from the photosensitive array and output the analog data to a backend processor. The image sensors of interest may be viewed as integrated circuit image sensors, and include complementary metal-oxide-semiconductor (CMOS) sensors and charge-coupled device (CCD) sensors. The image sensors may further include a lens positioned relative to the photosensitive component so as to focus images on the photosensitive component. A signal conductor may be present to connect the image sensor at the distal and to a device at the proximal end of the elongate member, e.g., in the form of one or more wires running along the length of the elongate member from the distal to the proximal end. Imaging sensors of interest include, but are not limited to, those obtainable from: OmniVision Technologies, Inc., Sony Corporations, Cypress Semiconductors, Aptina Imaging. As the imaging sensor(s) is integrated at the distal end of the tissue modification device, it cannot be removed from the remainder of the tissue modification device without significantly compromising the structure of the modification device. As such, the integrated visualization element is not readily removable from the remainder of the tissue modification device, such that the visualization element and remainder of the tissue modification device form an inter-related whole.

While any convenient imaging sensor may be employed in devices of the invention, in certain instances the imaging sensor is a CMOS sensor. Of interest as CMOS sensors are the OmniPixel line of CMOS sensors available from OmniVision (Sunnyvale, Calif.), including the OmniPixel, OmniPixel2, OmniPixel3, OmniPixel3-HS and OmniBSI lines of CMOS sensors. These sensors may be either frontside or backside illumination sensors, and have sufficiently small dimensions while maintained sufficient functionality to be positioned at the distal end of the minimally invasive devices of the invention. Aspects of these sensors are further described in one or more the following U.S. patents, the disclosures of which are herein incorporated by reference: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

In certain embodiments, the systems of the invention are used in conjunction with a controller configured to control illumination of the illumination elements and/or capture of images (e.g., as still imaged or video output) from the image sensors. This controller may take a variety of different formats, including hardware, software and combinations thereof. The controller may be physically located relative to the tissue modification device and/or access device at any convenient location, where the controller may be present at the distal end of the system components, at some point between the distal and proximal ends or at the proximal ends of the system components, as desired. In certain embodiments, the controller may be distinct from the system components, i.e., access device and tissue modification device, such the access device and/or elongated member includes a controller interface for operatively coupling to the distinct controller, or the controller may be integral with the device.

Systems of the invention may include a number of additional components in addition to the tissue modification and access devices as described above. Additional components may include root retractors, device fixation devices, image display units (such as monitors), data processors, e.g., in the form of computers, etc.

The devices or components thereof of the systems may be configured for one time use (i.e., disposable) or be re-usable, e.g., where the components are configured to be used two or more times before disposal, e.g., where the device components are sterilizable.

### Rongeur System Including Integrated Visualization Element

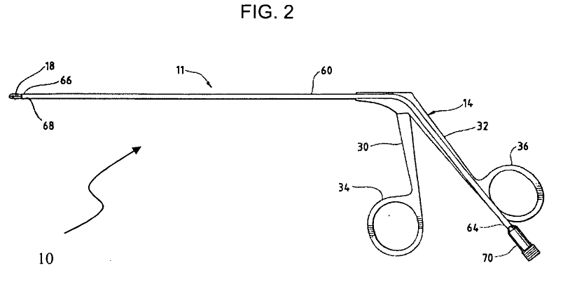

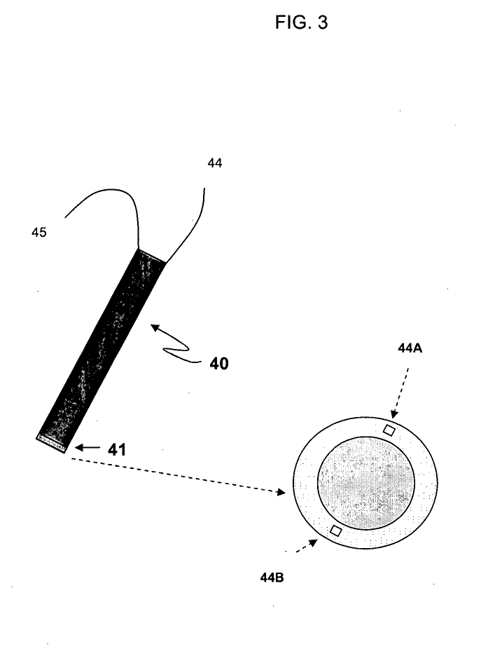

In certain instances, systems of the invention are minimally invasive rongeur systems. The term “rongeur” is employed in its conventional sense to refer to a forceps device configured to remove small pieces of bone or tough tissue. An illustration of a rongeur system according to an embodiment of the invention is depicted in FIGS. 2 and 3.

In FIG. 2, a rongeur device 10 in accordance an embodiment of the present invention is shown. Rongeur device 10 includes elongated member or shaft 11 having a handle 14 mounted on a proximal end 64 of the shaft, and a working element 18 mounted on a distal end 68 of the shaft. The surgical instrument 10 also includes a visualization element, such as a CMOS or CCD camera 66, integrated at the distal end 68 of the device and near to the working element 18. In certain instances, the image sensor may be integrated with the working element itself, such as a forceps member of the working element. The handle 14 has a portion that is intended to be gripped or held by a surgeon so that the working element can be used to manipulate tissue during a surgical procedure.

The handle 14 is offset relative to the shaft 11, and includes a first handle member 30 that is pivotally connected to a second handle member 32. The handle members 30 and 32 terminate in respective finger receiving loops 34 and 36. The handle members 30 and 32 and the loops 34 and 36 form the gripping portion of the handle 14. Also shown at distal end 64 is imaging device interface element 70, which may provide for operative coupling of a wire running the length of the device to monitor (not shown).

The working element 18 is rigidly secured to the distal end 68 of the shaft 11 in any suitable manner. While the working element 18 is in the form of forceps, the working element 18 instead, however, may include a scissors, knife, probe, or coagulator, electrosurgical electrodes, or any other suitable tool.

The shaft 11 may include a central lumen or tube with its proximal end fitted with an interface element 70 in the second handle member 32 (see, e.g., FIG. 2), which interface element 70 allows for operable connection of the integrated visualization element with an external image display unit. The shaft 11 may be straight or have a predetermined bend or curve along its axis. The shaft 11 may be rigid. It may be flexible, bendable or malleable so that it may be adjusted by the surgeon. For example, the shaft may have a distal portion that is displaceable to alternative positions wherein the distal portion does not have the same axis as a proximal portion of the shaft.

The shaft 11 may also include an actuating mechanism operably coupled to the working element 18 to operate the working element. An actuating rod or cable may be affixed to the upper end of the first handle member 30 and extend through a lumen defined by a tube in shaft 11 to join the movable forceps 18. The shaft 11 may be constructed of a stainless steel or any other suitable material.

With this embodiment, by grasping the handle members 30 and 32 by their respective finger-receiving loops 34 and 36, and by pivoting the first handle member 30 back and forth relative to the stationary second handle member 32, the rod or cable moves reciprocally within the tube to cause the forceps or working element 18 to open and close in a scissors-like action.

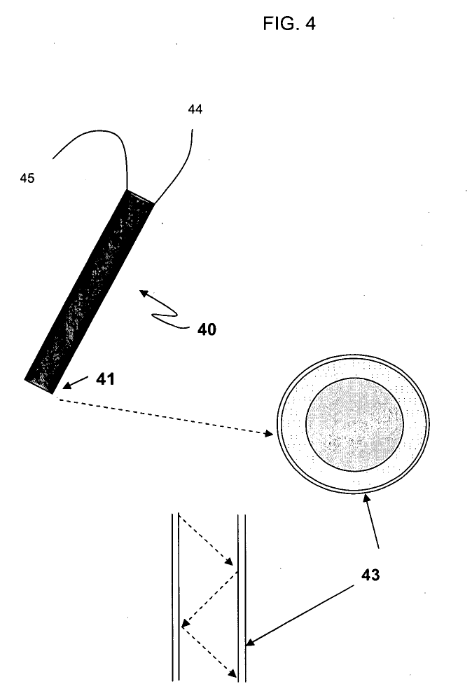

FIG. 3 provides different views of an access device according to an embodiment of the invention. As shown in FIG. 3, access device 40 includes a distal end 41. Positioned at distal end 41 are two illumination sources, e.g., LEDs or light fibers, 44A and 44B. Running the length of the access device and exiting the proximal end are wires 44 and 45 for providing power and control to the visualization elements, e.g., via coupling to a control device. FIG. 4 provides a view of an alternative embodiment of the device shown in FIG. 3, where the device is fabricated from a translucent material and includes an outer reflective coating 43 which guides light from the proximal end to the distal end 41. Inner surface of the device also includes a reflective coating to ensure that light can propagate from the proximal end to the distal end of the device

While the above description with respect to FIGS. 2 and 3 is specifically directed to rongeur systems of the invention, as illustrated above the systems of the invention are not so limited. Instead, systems of the invention include modified versions of any single port laparascopic device system which may include an access device and an instrument configured to be slidably introduced to a tissue location through the access device. Examples of such devices that may be modified to be systems of the invention (for example by including a visualization element on the instrument and an illumination source on the access device) include, but are not limited to: tissue sealers, graspers, dissectors, cautery devices and needle holders, e.g., as sold under the REALHAND™ product line by Novare Surgical Systems, Inc., Cupertino Calif.) and the ENDO AUTONOMY™ LAPARO-ANGLE CHECK product line from Cambridge Endo (Framingham, Mass.).

### Methods

Aspects of the invention further include methods of modifying an internal tissue site with the minimally invasive systems of the invention. A variety of internal tissue sites can be modified with devices of the invention. In certain embodiments, the methods are methods of modifying an intervertebral disc in a minimally invasive manner. For ease of description, the methods are now primarily described further in terms of modifying IVD target tissue sites. However, the invention is not so limited, as the devices may be used to modify a variety of distinct target tissue sites, including those listed above in the introduction section of the present application.

With respect to modifying an intervertebral disc or portion thereof, e.g., herniated portion of a disc, embodiments of such methods include positioning a distal end of a minimally invasive intervertebral disc modification device of the invention in viewing relationship to an intervertebral disc or portion of there, e.g., nucleus pulposus, internal site of nucleus pulposus, etc. By viewing relationship is meant that the distal end is positioned within 40 mm, such as within 10 mm, of the target tissue site of interest. Positioning the distal end in viewing device in relation to the desired target tissue may be accomplished using any convenient approach, including through use of an access device, such as a cannula or retractor tube, which may or may not be fitted with a trocar, as desired, where the access device is a device having illumination element (s) at its distal end. Following positioning of the distal end of the tissue modification device in viewing relationship to the target tissue, the target tissue, e.g., intervertebral disc or portion thereof, is imaged through use of the illumination and visualization elements to obtain image data. Image data obtained according to the methods of the invention is output to a user in the form of an image, e.g., using a monitor or other convenient medium as a display means. In certain embodiments, the image is a still image, while in other embodiments the image may be a video.

Following or during imaging, the methods include a step of tissue modification in addition to the tissue viewing. For example, the methods may include a step of tissue removal, e.g., using forceps of the device to grab and remove target tissue. For example, the methods may include grabbing a least a portion of the herniated tissue of a herniated disc and then removing the grabbed tissue from the site.

Methods of invention may find use in any convenient application, including diagnostic and therapeutic applications. Specific applications of interest include, but are not limited to, intervertebral disc diagnostic and therapeutic applications. For example, methods of the invention include, but are not limited to: annulotomy, nucleotomy, discectomy, annulus replacement, nucleus replacement, and decompression due to a bulging or extruded disc. Additional methods in which the imaging devices find use include those described in United States Published Application Nos. 20080161809; 20080103504; 20080051812; 20080033465; 20070213735; 20070213734; 20070123733; 20070167678; 20070123888; 20060258951; 2006024648; the disclosures of which are herein incorporated by reference.

Methods and devices of the invention may be employed with a variety of subjects. In certain embodiments, the subject is an animal, where in certain embodiments the animal is a “mammal” or “mammalian.” The terms mammal and mammalian are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects (i.e., patients) are humans.

### Kits

Also provided are kits for use in practicing the subject methods, where the kits may include one or more of the above devices, and/or components of the subject systems, as described above. As such, a kit may include a tissue modification device and an access device, as described above. The kit may further include other components, e.g., guidewires, stylets, tissue retractors, etc., which may find use in practicing the subject methods. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer Readable Storage Media

Also of interest is programming that is configured for operating a visualization device according to methods of invention, where the programming is recorded on physical computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of instructions for operating a minimally invasive of the invention.

Programming of the invention includes instructions for operating a device of the invention, such that upon execution by the programming, the executed instructions result in execution of the imaging device to: illuminate a target tissue site, such as an intervertebral disc or portion thereof; and capture one or more image frames of the illuminated target tissue site with the imaging sensor.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A minimally invasive tissue modification system, the system comprising:
(a) a minimally invasive access device having a proximal end, a distal end and an internal passageway; and
(b) an elongated tissue modification device having a proximal end and a distal end, wherein the tissue modification device is dimensioned to be slidably moved through the internal passageway of the access device;
wherein the system includes an illumination element and a visualization element positioned among the distal ends of the access device and tissue modification device.

2. The minimally invasive tissue modification system according to claim 1, wherein the illumination element comprises a LED.

3. The minimally invasive tissue modification system according to claim 1, wherein the illumination element comprises a fiber optic light source.

4. The minimally invasive tissue modification system according to claim 1, wherein the illumination element comprises both a LED and a fiber optic light source.

5. The minimally invasive tissue modification system according to claim 1, wherein the illumination element includes a diffusion element.

6. The minimally invasive tissue modification system according to claim 1, wherein the visualization element is selected from a CCD and a CMOS sensor.

7. The minimally invasive tissue modification system according to claim 6, wherein the visualization element is operably coupled to an image display unit at the proximal end of the tissue modification device.

8. The minimally invasive tissue modification system according to claim 1, wherein the tissue modifier is a mechanical tissue modifier.

9. The minimally invasive tissue modification system according to claim 8, wherein the tissue modification device is a rongeur.

10. The minimally invasive tissue modification system according to claim 9, wherein the visualization element is positioned at the distal tip of the rongeur.

11. A method of modifying an internal target tissue of a patient, the method comprising:
(a) positioning a minimally invasive access device having a proximal end, a distal end and an internal passageway so that the distal end is near the target tissue, wherein the distal end comprises an illumination element; and
(b) slidably moving an elongated tissue modification device having a proximal and distal end through the internal passageway of the access device so that the distal end is operably positioned in relation to the target tissue, wherein the tissue modification device includes a tissue modifier and a visualization element integrated at the distal end; and
(c) modifying the target tissue with the tissue modifier.

12-21. (canceled)

22. A kit comprising:
(a) a minimally invasive access device having a proximal end, a distal end and an internal passageway, wherein the distal end comprises an illumination element; and
(b) an elongated tissue modification device having a proximal end and a distal end, wherein the tissue modification is dimensioned to be slidably moved through the internal passageway of the access device and includes a visualization element at the distal end.

23. The kit according to claim 22, wherein the illumination element comprises a LED.

24. The kit according to claim 22, wherein the illumination element comprises a fiber optic light source.

25. The kit according to claim 22, wherein the illumination element comprises both a LED and a fiber optic light source.

26. The kit according to claim 22, wherein the illumination element comprises a diffusion element.

27-31. (canceled)

32. A minimally invasive access device having a proximal end, a distal end and an internal passageway, wherein the distal end comprises an illumination element.

33. The minimally invasive access device according to claim 32, wherein the illumination element comprises a LED.

34. The minimally invasive access device according to claim 32, wherein the illumination element comprises a fiber optic light source.

35. The minimally invasive access device according to claim 32, wherein the illumination element comprises both a LED and a fiber optic light source.

36. The minimally invasive access device according to claim 32, wherein the illumination element includes a diffusion element.
